(19) United States
(12) Patent Application Publication
(10) Pub. No.: US 2026/0224878 A1
(43) Pub. Date: Aug. 6, 2026

(54) HEMOSTASIS VALVES AND METHODS OF USE

(71) Applicant: Stryker Corporation, Portage, MI (US)

(72) Inventors: Brandon Conrad, Luguna Niguel, CA (US); Behraam Hussain Baqai, Irvine, CA (US); Sierra Copner, Costa Mesa, CA (US)

(21) Appl. No.: 19/466,171

(22) Filed: Jan. 30, 2026

Related U.S. Application Data
(60) Provisional application No. 63/752,086, filed on Jan. 31, 2025.

(51) Int. Cl. A61M 39/06

(52) U.S. Cl. CPC A61M 39/0613; A61M 2039/062; A61M 2039/0633

(57) ABSTRACT

A hemostatic valve for sealing a medical device includes a unibody elongate member having a first end, a second end, and a central lumen extending therebetween, wherein the unibody elongate member is pliable. The hemostatic valve includes a reinforcement structure extending along at least a portion of the unibody elongate member, wherein the reinforcement structure is coupled to the unibody elongate member. An active tensioning mechanism including an actuator coupled to the unibody elongate member, wherein the actuator is moveable between (a) a first position wherein the central lumen is constricted and sealed, and (b) a second position wherein the central lumen is open, and wherein the tensioning mechanism includes at least one filament extending at least partially around the unibody elongate member. The hemostatic valve includes a biasing member configured to bias the actuator to the first position.

### CROSS-REFERENCE TO RELATED APPLICATION

This application claims the benefit of U.S. Provisional Patent Application No. 63/752,086, filed Jan. 31, 2025, and titled “HEMOSTASIS VALVES AND METHODS OF USE,” which is incorporated herein by reference in its entirety.

### TECHNICAL FIELD

The present technology generally relates to hemostasis valves, and more particularly, to hemostasis valves including a unibody septum.

### BACKGROUND

During a surgical procedure, a portion of a patient's body (e.g., vasculature) is accessed to allow for the performance of a desired intervention or treatment. During such surgical procedures, it is desired to minimize patient blood loss, prevent delivery of air into the vasculature, and to maintain the sterility of the accessed portions or sites of the patient's body so as to prevent issues such as infection. Further, the desire for improved patient outcomes has led to the development of hemostasis valves that facilitate minimally invasive surgery.

In minimally invasive surgery, small incisions are created through a blood vessel which one or several catheters are inserted. Each of these one or several catheters can define a lumen extending longitudinally through that catheter. These catheters are moved to a position proximate to tissue, nerves, or other body structures targeted by the surgery, and then tools for performing the procedure are inserted through the lumens of some or all of these catheters.

To minimize blood loss, prevent delivery of air into the vasculature, and to facilitate maintenance of sterility within the patient's body (e.g., blood vessel), these catheters are equipped with hemostasis valves. These valves seal or selectably seal the lumens of the catheters. In many instances, these valves can seal the lumen of the catheter when a tool extends through the catheter, and specifically through the valve. Additionally, the valves can seal the lumen when a tool is removed or does not extend through the catheter.

While such traditional hemostasis valves are greatly beneficial for intravascular access, they have some drawbacks. For example, some valves may not seal adequately for all interventional applications or tools, and/or the operation of some valves may be complicated for operator use. The drawbacks of such valve designs may in turn increase the complexity of any surgery performed therewith and/or reduce patient safety (e.g., bleeding, infection, and/or other detrimental complications). Accordingly, new and improved hemostasis valves and methods of use are desired.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a perspective view of a delivery device in accordance with embodiments of the present technology.

FIG. 2 is a side-section view of a hemostasis valve in a first configuration in accordance with embodiments of the present technology.

FIG. 3 is a side-section view of the valve in a second configuration in accordance with embodiments of the present technology.

FIG. 4 is a side-section view of the valve in the first configuration and with a tool extending through the valve in accordance with embodiments of the present technology.

FIG. 5 is a perspective view of a filament of a valve forming a loop in accordance with embodiments of the present technology.

FIG. 6 is a perspective view of two filaments of a valve, each of the filaments forming a loop in accordance with embodiments of the present technology.

FIG. 7 is a perspective view of two overlapping and interlocked bights in an open configuration in accordance with embodiments of the present technology.

FIG. 8 is a perspective view of two overlapping and interlocked bights in a closed configuration in accordance with embodiments of the present technology.

FIG. 9 is a flowchart of a method for sealing a valve and/or catheter in accordance with embodiments of the present technology.

FIG. 10 is a side view of a thrombectomy system including the delivery device in accordance with additional embodiments of the present technology.

FIG. 11 is a cross-sectional view of an elongate member of the hemostasis valve of FIG. 2 in accordance with additional embodiments of the present technology.

FIG. 12 is a side-section view of a hemostasis valve in a first configuration in accordance with additional embodiments of the present technology.

FIG. 13 is a cross-sectional view of an elongate member of the hemostasis valve of FIG. 12 in accordance with additional embodiments of the present technology.

FIG. 14 is a side-section view of a hemostasis valve in a second configuration in accordance with additional embodiments of the present technology.

FIG. 15 is a cross-sectional view of an elongate member of the hemostasis valve of FIG. 14 in accordance with additional embodiments of the present technology.

### DETAILED DESCRIPTION

### I. Introduction

The following relates to valves, medical systems incorporating valves, and methods of using the same. The valve can include a tubular member that can be constricted, collapsed, and/or sealed by one or several tensioning mechanisms. The tensioning mechanism can include at least one filament that extends around at least a portion of the tubular member. The filament can interact with the tubular member to constrict, collapse, and/or seal the tubular member via manipulation of the tensioning mechanism(s). A tool can be inserted through the valve to gain access to a patient's body, and specifically to gain access to a blood vessel. Through the use of the tensioning mechanism and filament to constrict, collapse, and/or seal the tubular member, the valve can seal around a wide range of tool sizes and shapes, as well as multiple tools of differing sizes simultaneously. Additionally, such a valve creates a robust seal that maintains its seal when a vacuum is applied, such as occurs during aspiration.

Aspects of the present disclosure relate to a hemostatic valve for sealing a medical device. The hemostatic valve includes a unibody elongate member having a first end, a second end, and a central lumen extending therebetween. The first end can include a proximal sealing feature, and the second end can include a distal sealing feature. In some embodiments, the unibody elongate member is pliable, such that it can be deformed (e.g., compressed, restricted) along a portion of or an entire length of the unibody elongate member. The hemostatic valve can optionally include a reinforcement structure extending along at least a portion of the unibody elongate member, such that the reinforcement structure is coupled to the elongate member. The hemostatic valve includes an active tensioning mechanism coupled to the elongate member. In some embodiments, the tensioning mechanism is moveable between a first configuration in which the central lumen is constricted and sealed and a second configuration in which the central lumen is open. In some embodiments, the valve can be manually adjusted by the user to intermediate positions between fully open and fully closed. Additionally, an instrument (e.g. catheter) can provide an intermediate position where the valve creates hemostasis without user adjustment.

In some embodiments, the elongate member can be a compliant polymer tube. In some embodiments, the tensioning mechanism can include at least one filament extending at least partially around the elongate member. In some embodiments, the reinforcement structure is positioned between the at least one filament and the elongate member. In some embodiments, the reinforcement structure can be a braided mesh. In some embodiments, the reinforcement structure is coupled to the elongate member at a position proximate to the first end of the elongate member and at a position proximate to the second end of the elongate member. In some embodiments, the reinforcement structure is not coupled to the elongate member at a position between the first end of the elongate member and the second end of the elongate member. In some embodiments, the central portion of the compliant polymer tube that is constrained or collapsed by the tensioning mechanism, and at least one filament, is not coupled to the reinforcement structure.

In some embodiments, the tensioning mechanism can include an actuator coupled to the at least one filament. In some embodiments, there are two tensioning mechanisms coupled to the at least one filament that operate in opposite directions. In some embodiments, the two tensioning mechanisms are attached to the same filament. In some embodiments, the two tensioning mechanisms are attached to opposing filaments. In some embodiments, the actuator can be moveable to control movement of the at least one filament from a first position in which the central lumen is constricted and sealed to a second position in which the central lumen is open. In some embodiments, the at least one filament is in the first position when the tensioning mechanism is in the first configuration. In some embodiments, the actuator is biased toward the first position. In some embodiments, the actuator is biased toward the second position. In some embodiments, the actuator can be a manual actuator.

In some embodiments, the hemostatic valve can include a shell defining a first aperture and a second aperture. In some embodiments, the elongate member extends from the first aperture to the second aperture and fluidly couples the first aperture and the second aperture. In some embodiments, the tensioning mechanism is self-adjustable to seal around tools of different sizes extending through the hemostatic valve. In some embodiments, the central lumen can comprise a single lumen, and in some embodiments, the central lumen can comprise a plurality of lumens.

One aspect of the present disclosure relates to a delivery system for intravascular access of a blood vessel within a patient's body. The delivery system includes a catheter having a first end, a second end, and a catheter lumen extending therebetween and a hemostatic valve coupled to the first end of the catheter. The hemostatic valve includes a unibody tubular member having a first end, a second end, and a central lumen extending therebetween. In some embodiments, the central lumen of the tubular member is fluidly coupled with the catheter lumen. The hemostatic valve includes an active tensioning mechanism coupled to the tubular member, the tensioning mechanism can be moveable between a first configuration in which the tensioning mechanism constricts on the central lumen and the central lumen is sealed, and a second configuration in which the central lumen is open.

In some embodiments, the hemostatic valve further includes a reinforcement structure extending along at least a portion of the tubular member. In some embodiments, the reinforcement structure is located between the tensioning mechanism and the tubular member. In some embodiments, the reinforcement structure can be a braided mesh. In some embodiments, the reinforcement structure is coupled to the tubular member at a position proximate to the first end of the tubular member and at a position proximate to the second end of the tubular member. In some embodiments, the reinforcement structure is adhered to the tubular member at the first end of the tubular member and at the second end of the tubular member. In some embodiments, the reinforcement structure is uncoupled to the tubular member between the first end of the tubular member and the second end of the tubular member.

In some embodiments, the tensioning mechanism can include at least one filament extending at least partially around the tubular member. In some embodiments, the tensioning mechanism can include an actuator coupled to the at least one filament. In some embodiments, moving the tensioning mechanism from the first configuration to the second configuration can include moving the actuator and the thereto coupled at least one filament from a first position to a second position. In some embodiments, the filament constricts and seals the central lumen of the tubular member when the filament is in the first position.

In some embodiments, the actuator can be a manual actuator. In some embodiments, the actuator can include a pair of opposing and depressable buttons, which can be biased towards an undepressed position. In some embodiments, the central lumen is sealed when the buttons are in the undepressed position. In some embodiments, the filament can be a monofilament. In some embodiments, the filament can be at least one of: a polymer filament, or a metallic filament. Regardless, the filament comprises a thin, flexible length of material formed by one or more strands of material. In some embodiments, the catheter can include a thrombus extraction device.

One aspect of the present disclosure relates to a method of sealing a delivery device accessing a blood vessel of a patient. The method includes inserting the delivery device, including a catheter and a hemostatic valve, into the blood vessel of the patient. In some embodiments, the catheter can have a first end, a second end, and a catheter lumen extending therethrough. In some embodiments, the hemostatic valve can be coupled to the first end and can have a tubular member defining a central lumen fluidly coupled with the catheter lumen and a tensioning mechanism coupled with the tubular member. In some embodiments, the tensioning mechanism collapses and seals the central lumen in a first configuration and thereby seals access to the blood vessel. The method can include moving the tensioning mechanism of the hemostatic valve to a second configuration. In some embodiments, the central lumen is open and access to the blood vessel is unsealed when the tensioning mechanism is in the second configuration. The method can include advancing a shaft of a tool through the delivery device until a first end of the tool reaches a desired position within the blood vessel of the patient and a portion of the shaft is positioned within the central lumen of the tubular member. The method can include returning the tensioning mechanism of the hemostatic valve to the first configuration such that the tubular member collapses on the shaft of the tool and seals around the shaft of the tool.

In some embodiments, the method includes retracting the shaft of the tool from the delivery device. In some embodiments, the tensioning mechanism is maintained in the first configuration during and after the retracting of the shaft of the tool from the delivery device. In some embodiments, the tensioning mechanism is moved to the second configuration during the retracting of the shaft of the tool from the delivery device, and the tensioning mechanism is returned to the first configuration after the shaft of the tool is retracted from the delivery device.

In some embodiments, the tensioning mechanism can include at least one filament extending at least partially around the tubular member. In some embodiments, the at least one filament collapses the tubular member when the tensioning mechanism is in the first configuration. In some embodiments, the at least one filament circumferentially constricts the tubular member to collapse the tubular member when the tensioning mechanism is in the first configuration. In some embodiments, the hemostatic valve can include a reinforcement structure located between the at least one filament and the tubular member.

In some embodiments, the at least one filament forms a loop around the elongate member, and moving the tensioning mechanism from the second configuration to the first configuration reduces a size of the loop to thereby constrict the tubular member within the loop. In some embodiments, the filament forms at least one bight around a portion of the elongate member. In some embodiments, the filament can include a first filament and a second filament. In some embodiments, the at least one bight can include a first bight oriented in a first direction and formed by the first filament and a second bight oriented in a second direction and formed by the second filament. In some embodiments, the first and second bights overlap to encircle a portion of the tubular member within a constricting area.

In some embodiments, moving the tensioning mechanism from the second configuration to the first configuration can include moving the first bight in the first direction and the second bight in the direction to reduce the size of the constricting area and collapse and seal the central lumen of the tubular member. In some embodiments, the tensioning mechanism can include an actuator. In some embodiments, moving the tensioning mechanism to the second configuration can include manipulating the actuator. In some embodiments, the method includes applying a vacuum to the delivery device and/or delivery system to aspirate material through the catheter. In some embodiments, the central lumen remains sealed during the aspiration. In some embodiments, the tool can include a thrombus extraction device.

In one aspect, a hemostatic valve for sealing a medical device includes a unibody elongate member having a first end, a second end, and a central lumen extending therebetween, wherein the unibody elongate member is pliable. The hemostatic valve includes a reinforcement structure extending along at least a portion of the unibody elongate member, wherein the reinforcement structure is coupled to the unibody elongate member. An active tensioning mechanism including an actuator coupled to the unibody elongate member, wherein the actuator is moveable between (a) a first position wherein the central lumen is constricted and sealed and (b) a second position wherein the central lumen is open, and wherein the tensioning mechanism includes at least one filament extending at least partially around the unibody elongate member. The hemostatic valve includes a biasing member configured to bias the actuator to the first position.

In another aspect, a hemostatic valve for sealing a medical device includes a unibody elongate member having a first end, a second end, and a central lumen extending therebetween, wherein the elongate member is pliable. The hemostatic valve includes a reinforcement structure extending along at least a portion of the unibody elongate member, wherein the reinforcement structure is coupled to the unibody elongate member. The hemostatic valve includes an active tensioning mechanism including an actuator coupled to the unibody elongate member and at least one filament coupled to the actuator and extending at least partially around the unibody elongate member, wherein the actuator is moveable between (a) a first position wherein the central lumen is constricted and sealed and (b) a second position wherein the central lumen is open, and wherein, in the first position, the actuator pulls the at least one filament to collapse the unibody elongate member such that the central lumen is constricted and sealed. The hemostatic valve includes a biasing member configured to bias the actuator to the first position.

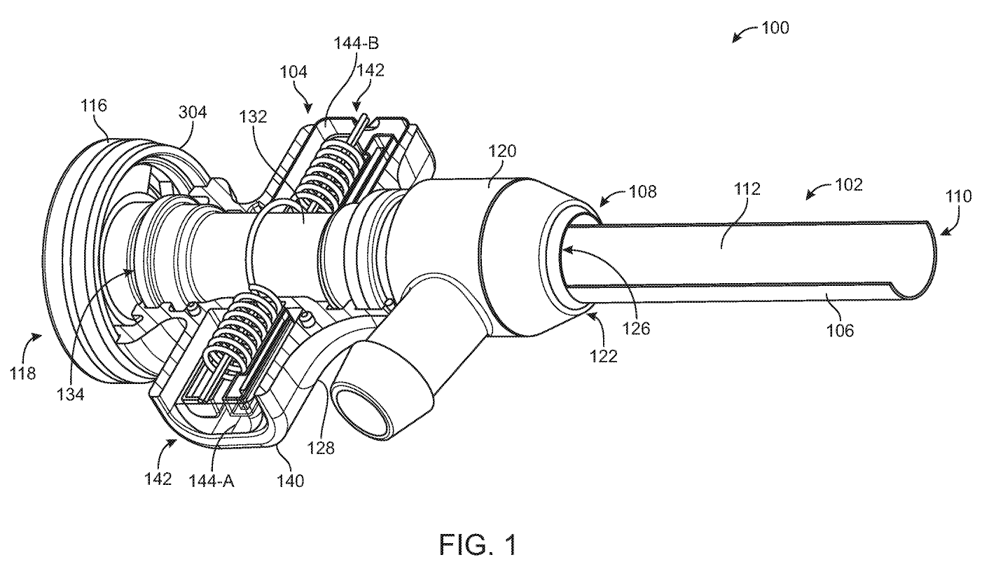

Certain details are set forth in the following description and in FIGS. 1-15 to provide a thorough understanding of various embodiments of the present technology. In other instances, well-known structures, materials, operations, and/or systems often associated with intravascular procedures, clot removal procedures, clot treatment systems, clot treatment devices, fluid control devices, syringes, blood-filtering syringes, catheters, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as the position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. The headings provided herein are for convenience only and should not be construed as limiting the subject matter disclosed. To the extent any materials incorporated herein by reference conflict with the present disclosure, the present disclosure controls.

### II. Selected Embodiments of Hemostasis Valves

The present disclosure relates to a valve that can be used as a hemostasis valve. This valve, also referred to herein as a garrote valve, can seal with or without a tool extending through the valve. The garrote valve provides convenient, single-handed operation for a wide range of medical devices, including catheters, wires, embolectomy systems, or the like. This single-handed operation of the garrote valve allows the user to easily and quickly swap different tools being used through the valve without compromising hemostasis and, therefore, simplifying the procedure. Combined with single-handed operation, the garrote valve provides robust sealing either with or without a tool extending through the valve. This robust sealing minimizes leakage in applications with a pressure differential on different sides of the valve. This pressure differential can arise, for example, during the application of vacuum aspiration in a procedure. Even under such conditions, as well as under other conditions, the garrote valve maintains seal integrity and inhibits, or even prevents, leakage in one or both directions. Example hemostasis valves are described in U.S. Patent Application No. 16/117,519, filed Aug. 30, 2018, granted May 11, 2021, as U.S. Patent No. 11,000,682, and titled “HEMOSTASIS VALVES AND METHODS OF USE,” each of which is incorporated herein by reference in its entirety.

The garrote valve includes a tubular member. The tubular member is a flexible member that defines a central lumen, which can, in some embodiments, define a single lumen, and in some embodiments, defines a plurality of lumens. In some embodiments, each of the plurality of lumens can comprise the same size and shape, and in some embodiments, some or all of the plurality of lumens can comprise different sizes and shapes. In some embodiments, for example, the plurality of lumens can comprise a lumen sized and/or shaped to receive a guide wire and a lumen sized and/or shaped to receive a tool. The tubular member extends at least partially through a constricting mechanism. The constricting mechanism can be moved from a first configuration to a second configuration, and the constricting mechanism can collapse and/or seal the central lumen of the tubular member when the constricting mechanism is in the first configuration. The constricting mechanism creates the above-discussed robust seal of the tubular member and thus of the valve.

With reference now to FIG. 1, a perspective view of one embodiment of a delivery system 100, also referred to herein as a delivery device 100, is shown. The delivery system 100 can include a catheter 102 and a garrote valve 104, also referred to herein as valve 104. The catheter 102 can comprise a shaft 106, also referred to herein as an elongate sheath 106, having a proximal end 108, also referred to herein as a first end 108, that can connect to the valve 104 and a distal end 110, also referred to herein as a second end 110. The shaft 106 can define a catheter lumen 112 extending from the proximal end 108 of the shaft 106 to the distal end 110 of the shaft 106. The catheter 102 and specifically the shaft 106 can comprise a variety of shapes and sizes and can be made from a variety of materials. In some embodiments, the catheter 102 can be flexible and/or can be made from a biocompatible material. The elongate sheath 106 can have size of at least 4 French, at least 6 French, at least 8 French, at least 10 French, at least 12 French, at least 14 French, at least 18 French, at least 20 French, at least 22 French, between 4 French and 30 French, between 8 French and 24 French, between 12 French and 20 French, and/or any other or intermediate size.

The valve 104 can include an outer shell 114. The outer shell 114 can comprise a variety of shapes and sizes and can be made from a variety of materials. In some embodiments, the outer shell 114 can be made from one or several polymers or composites. The outer shell 114 can include features that allow interaction with and/or control of the valve 104 to move the valve 104 between the first configuration and the second configuration.

The outer shell 114 can include a proximal cap 116 located at a proximal end 118 of the outer shell 114 and a distal cap 120 located at a distal end 122 of the outer shell 114. The proximal cap 116 can include and/or house a proximal aperture 124, also referred to herein as a proximal channel 124, a first channel 124, or a first aperture 124, that extends through the proximal cap 116, and the distal cap 120 can include and/or house a distal aperture 126, also referred to herein as a distal channel 126, a second channel 126, or second aperture 126, that extends through the distal cap 120. As seen in FIG. 1, the distal cap 120 connects to the shaft 106 of the catheter 102 at the distal end 122 of the valve 104.

The proximal cap 116 and the distal cap 120 are connected via a housing 128. The housing 128 can be a one-piece housing 128 or a multi-piece housing 128. In the embodiment depicted in FIG. 1, the housing includes a two-piece housing 128. The housing 128 can be configured to receive and couple with each of the proximal cap 116 and the distal cap 120, and as seen in FIG. 1, the housing 128 is coupled with each of the proximal cap 116 and the distal cap 120 to secure the relative position of the proximal cap 116 and the distal cap 120 with respect to each other.

The housing 128 can define an interior channel 130 through which a unibody elongate member 132 (which can also referred to as an elongate member, a tubular member, a septum, a tubular septum, a tube, a channel, and/or the like) can extend and connect the proximal cap 116 and the distal cap 120. The elongate member 132 can include a variety of shapes and sizes and be manufactured from a variety of materials. The interior channel 132 and/or the elongate member 132 can have a size the same as, smaller than, or larger than the elongate sheath 106. In some embodiments, the elongate member 132 can define a compliant tubular structure, for example, a thin-walled compliant tubular structure. The thin-walled structure of the elongate member 132 can facilitate the collapse, and specifically the uniform collapse of at least a portion of the elongate member 132 and the sealing of the elongate member 132. In some embodiments, the elongate member 132 is an elastic, resilient material that can include a polymer, including a natural and/or synthetic polymer. In some embodiments, the elongate member can include an elastic, resilient material that can include silicone, urethane, ethylene-vinyl acetate, natural or synthetic rubber, or other elastomers known in the art. In some embodiments, the elongate member 132 can include a silicone tube.

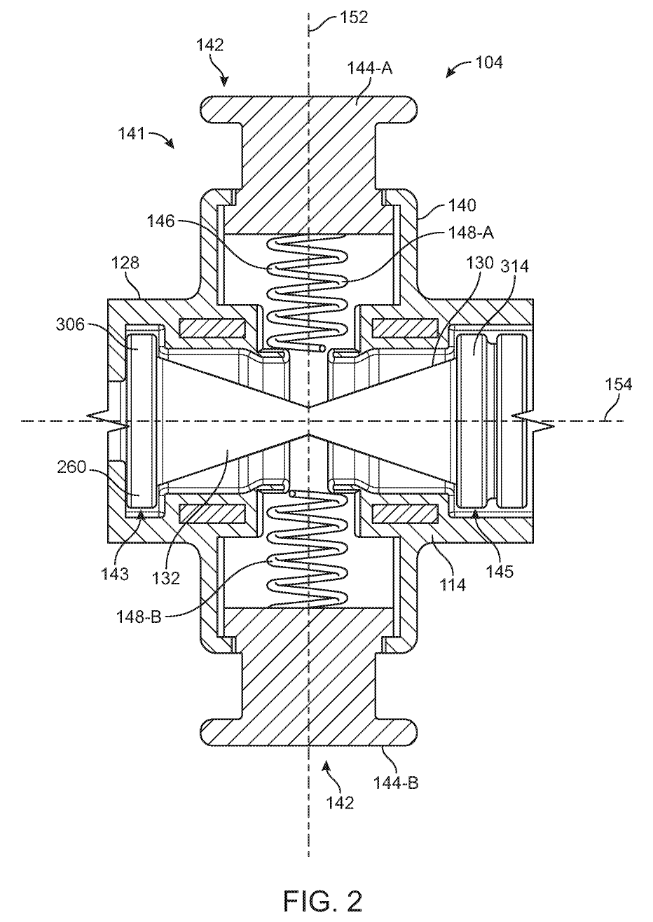

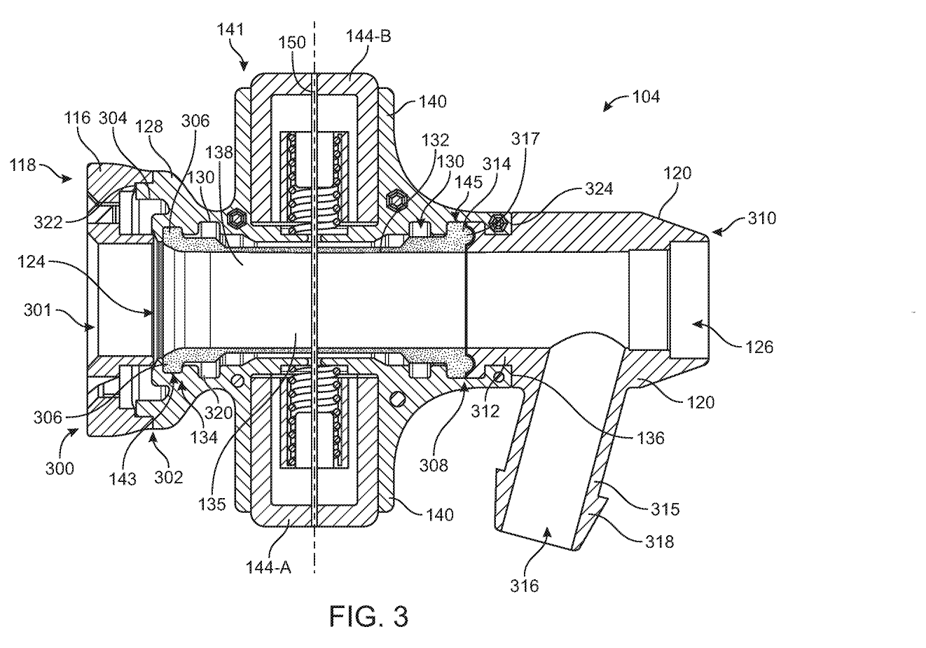

The elongate member 132 can include a proximal end 134, also referred to herein as a first end 134, that can couple to the proximal cap 116, and a distal end 136, also referred to herein as a second end 136, that can couple to the distal cap 120. The elongate member 132 can define a central lumen 138 that can extend from the first end 134 to the second end 136 of the elongate member 132. The elongate member 132 can be coupled to the proximal cap 116 such that the central lumen 138 is fluidly coupled with the proximal aperture 124 of the proximal cap 116, and the elongate member 132 can be coupled to the distal cap 120 such that the central lumen 138, as seen in FIG. 2 and in FIG. 3, is fluidly coupled with the distal aperture 126 of the distal cap 120.

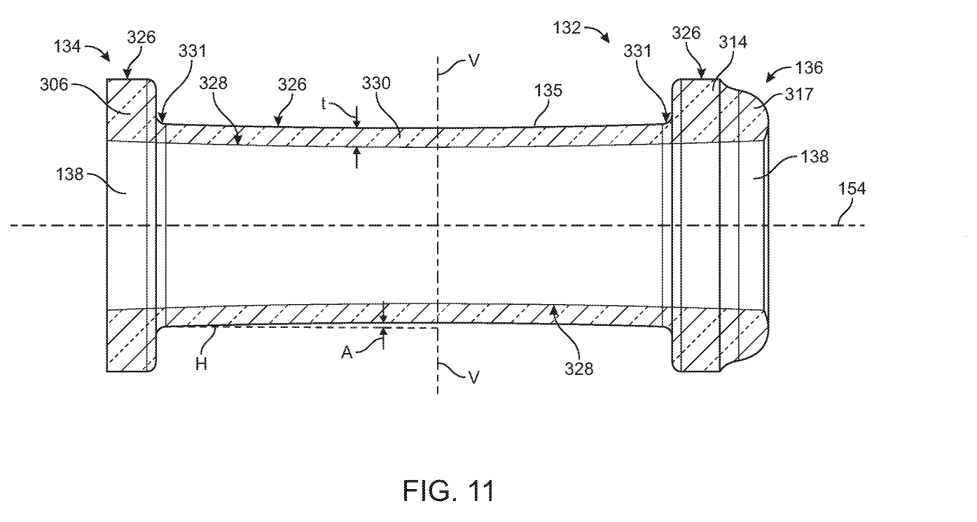

The central lumen 138 of the elongate member 132 can be defined by a wall 330 (FIGS. 11, 13, and 15) of the elongate member 132 that can have a thickness (t) that is uniform along the length of the elongate member 132 between the first end 134 and the second end 136, or that is non-uniform along the length of the elongate member 132 between the first end 134 and the second end 136. In some embodiments, the wall can have a thickness (t) that is approximately between about 0.005 inches and about 0.05 inches, and/or between about 0.010 inches and about 0.030 inches.

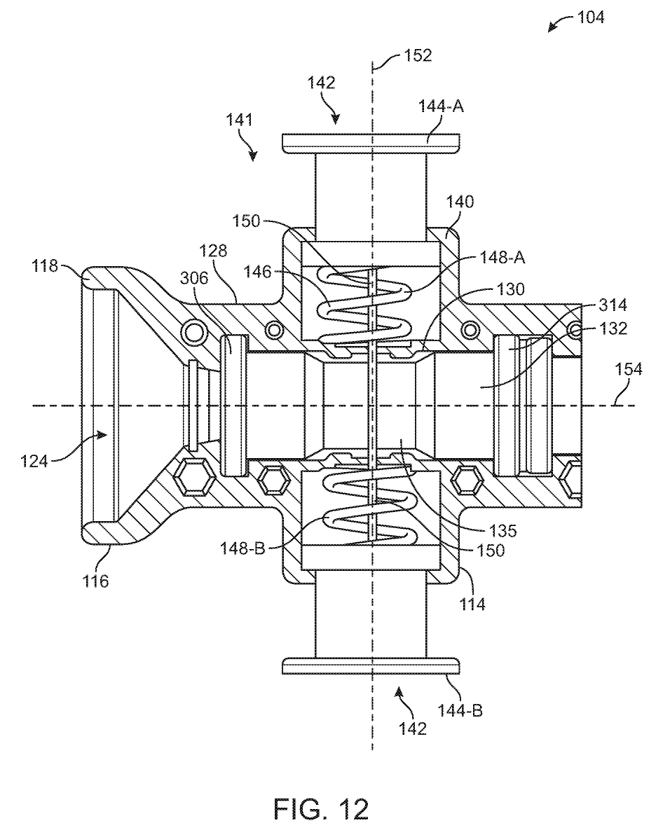

In some embodiments, the elongate member 132 can be cylindrically shaped, and specifically can be circular-cylindrically shaped. In some embodiments, the elongate member 132 can be dog-bone shaped to facilitate, for example, connection to each of the proximal cap 116 and the distal cap 120. In some embodiments, the elongate member 132 can be straight tapered in the direction of an axis 152 positioned between the proximal cap 116 and the distal cap 120 (FIGS. 2 and 11). In some embodiments, the elongate member 132 can be step-tapered in the direction of the axis 152 (FIGS. 12-15). In some embodiments, the elongate member 132 can include one or several outward-extending protuberances that engage with all or portions of a constricting mechanism 141, also referred to herein as a tensioning mechanism 141, of the valve 104 to secure a position of all or portions of the constricting mechanism 141 with respect to the elongate member 132. In some embodiments, the constricting mechanism 141 can be self-adjusting to seal around tools of different sizes extending through the valve 104.

Referring to FIG. 2, the proximal end 134 of the elongate member 132 defines a proximal sealing feature 306, and the distal end 136 of the elongate member 132 defines a distal sealing feature 314. The proximal sealing feature 306 and the distal sealing feature 314 of the elongate member 132 are spaced by a distance from each other along a central axis 154. Referring to FIGS. 2 and 11 together, the proximal sealing feature 306 can be spaced a first distance from an axis (V) of the elongate member 132 that is orthogonal to the central axis 154, and the distal sealing feature 314 can be spaced a second distance from the axis (V). In some instances, the first and second distances are about the same. In other instances, the first and second distances are different from each other, such that the first distance is greater than the second distance or the second distance is greater than the first distance. The axis (V) can designate the central portion of the elongate member 132. However, it should be understood that the axis (V) is illustrated to aid in the description of the elongate member 132 and is not intended to be limiting in any manner.

In some aspects of the present technology, the proximal sealing feature 306 of the elongate member 132 and/or the distal sealing feature 314 of the unibody elongate member 132 can be configured to maintain the position of the elongate member 132 relative to the housing 128. For example, the proximal and distal sealing features 306, 314 can each be sized and shaped to be positioned in proximity to and/or at least partially within a corresponding sealing feature of the housing 128 (e.g., a groove or channel within the housing 128). More specifically, the proximal sealing feature 306 can be sized and shaped to be positioned in proximity to and/or at least partially within a corresponding proximal sealing feature 143 of the housing 128. The distal sealing feature 314 can be sized and shaped to be positioned in proximity to and/or at least partially within a corresponding distal sealing feature 145 of the housing 128. In some embodiments, the proximal sealing feature 306 and/or the distal sealing feature 314 can each include one or more flanges 260 (e.g., ridges) that extend into the proximal sealing feature 143 or the distal sealing feature 145, respectively. In the illustrated embodiment, for example, the proximal sealing feature 306 includes one flange 260 and the distal sealing feature 314 includes two flanges 260.

The elongate member 132 can be sized and shaped to fit within the interior channel 130 of the housing 128. The interior channel 130 can define corresponding features of the elongate member 132 such that the cross-sectional shape of the interior channel 130 at least partially resembles (e.g., complements) the cross-sectional shape of the elongate member 132. When assembled, the axis (V) of the elongate member 132 can be positioned in proximity to the axis 152 of the valve 104. In some embodiments, an outer diameter of the proximal sealing feature 306 and/or the distal sealing feature 314 can be greater than an inner diameter of the interior channel 130 of the housing 128.

Referring to FIGS. 2, 11, 13, and 15 together, the elongate member 132 can define a surface sealing feature 317 positioned on or adjacent to the distal sealing feature 314 and/or the proximal sealing feature 306. The surface sealing feature 317 can extend outwardly along the central axis 154 in a direction away from the proximal sealing feature 306 and/or the distal sealing feature 314. The surface sealing feature 317 can be positioned to seal against a surface of an adjacent component. For example, the surface sealing feature 317 can be positioned to seal against a surface of the distal cap 120, as shown in at least FIG. 3. In some embodiments, the surface sealing feature 317 can define a rounded cross-sectional shape, as shown in FIGS. 11, 13 and 15, and, in other embodiments, the surface sealing feature 317 can define a rectangular, oval, or other cross-sectional shape complementary to the adjacent component.

Referring to FIGS. 2 and 3 together, the elongate member 132 defines a midsection 135 that is situated between the proximal sealing feature 306 and the distal sealing feature 314 along the central axis 154. The wall 330 of the elongate member 132 defines an outer surface 326 and an inner surface 328 spaced from the outer surface 326 in the direction of the central axis 154. The distance between the outer surface 326 and the inner surface 328 of the wall 330 can define various thicknesses (t) depending, in part, on the position along the central axis of the elongate member 132. For example, the thickness (t) of the wall 330 of the proximal sealing feature 306 and the wall 330 of the distal sealing feature 314 can be greater than the thickness (t) of the wall 330 of the midsection 135. In some instances, as will be discussed in more detail below, the thickness (t) of the midsection 135 can vary along the central axis 154. The central lumen 138 is at least partially defined by the inner surface 328 and extends through at least the proximal sealing feature 306, the midsection 135, and the distal sealing feature 314.

In some embodiments and referring to FIGS. 2 and 11 together, the elongate member 132 defines a straight tapered body having a cross-sectional shape that tapers from a larger width in proximity to the proximal end 134 of the midsection 135 and tapers to a smaller width in proximity to the axis (V) of the midsection 135. Similarly, the elongate member 132 defines a cross-sectional shape that tapers from a larger width in proximity to the distal end 136 of the midsection 135 and tapers to a smaller width in proximity to the axis (V) of the midsection 135. Referring to FIG. 11, beginning at the proximal end 134 of the midsection 135 and moving in the direction toward the axis (V) along the central axis 154, the wall 330 of the midsection 135 can be tapered at an angle (A) relative to axis (H). Referring to FIG. 11, beginning at the distal end 136 of the midsection 135 and moving in the direction toward the axis (V) along the central axis 154, the wall 330 of the midsection 135 can be tapered at the angle (A) relative to axis (H). Axis (H) is parallel to the central axis 154. The straight tapered elongate member 132 transitions from the proximal sealing feature 306 to the midsection and from the distal sealing feature 314 to the midsection 135. The straight tapered elongate member 132 can transition from the proximal and distal sealing features 306, 314 to the midsection 135 by way of a fillet 331 or similarly shaped feature 331 to provide a gradual transition. The straight tapered elongate member 132 can further gradually increase in thickness from the axis (V) along the central axis 154 to the distal end 136. As illustrated, the midsection 135 of the elongate member 132 can decrease in thickness from the proximal end 134 along the central axis 154 up until the axis (V) and then increase in thickness along the central axis 154 up until the distal end 136.

Referring to the cross-sectional view of FIG. 11, an outer diameter of the proximal sealing feature 306 can be greater than or at least equal to an outer diameter of the midsection 135. Referring to the cross-sectional view of FIG. 11, an outer diameter of the distal sealing feature 314 can be greater than or at least equal to the outer diameter of the midsection 135. In some embodiments, an inner diameter of the proximal sealing feature 306 and/or the distal sealing feature 314 (e.g., a diameter of the central lumen 138) can be greater than or at least equal to an inner diameter of the midsection 135.

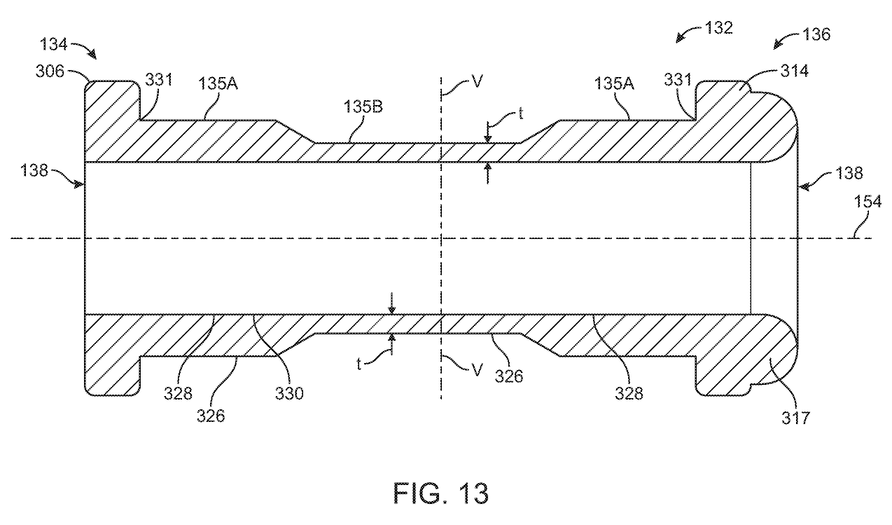

In another embodiment and referring to FIGS. 12 and 13 together, the elongate member 132 defines a step-tapered body having a cross-sectional shape that includes a first width in proximity to the proximal end 134 of the midsection 135, hereinafter referred to as the first midsection 135A, and extending in the direction of the axis (V). The elongate member 132 transitions to a second width in proximity to the midsection 135, hereinafter referred to as the second midsection 135B. Similarly, the elongate member 132 defines a cross-sectional shape that the first width in proximity to the distal end 136 of the first midsection 135A and extending in the direction of the axis (V). The elongate member 132 transitions to the second width in proximity to the second midsection 135B. The second width of the second midsection 135B is smaller than the first width of the first midsection 135A. The first midsection 135A can transition to the second midsection 135B at an angled tapered portion. The first midsection 135A and/or the second midsection 135B can be straight, tapered, or partially straight and partially tapered. The step-tapered elongate member 132 transitions from the proximal sealing feature 306 to the midsection 135 and from the distal sealing feature 314 to the first midsection 135A. The step-tapered elongate member 132 can transition from the proximal and distal sealing features 306, 314 to the first midsection 135A by way of a fillet 331 or similarly shaped feature 331 to provide a gradual transition.

Referring to the cross-sectional view of FIG. 13, an outer diameter of the proximal sealing feature 306 can be greater than or at least equal to an outer diameter of the first midsection 135A. Referring to the cross-sectional view of FIG. 13, an outer diameter of the distal sealing feature 314 can be greater than or at least equal to the outer diameter of the first midsection 135A. In some embodiments, an inner diameter of the proximal sealing feature 306 and/or the distal sealing feature 314 (e.g., a diameter of the central lumen 138) can be greater than or at least equal to an inner diameter of the first midsection 135A.

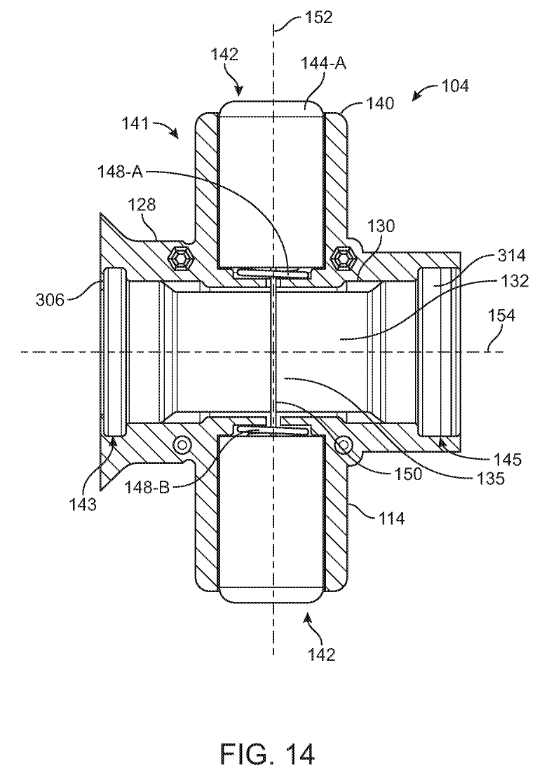

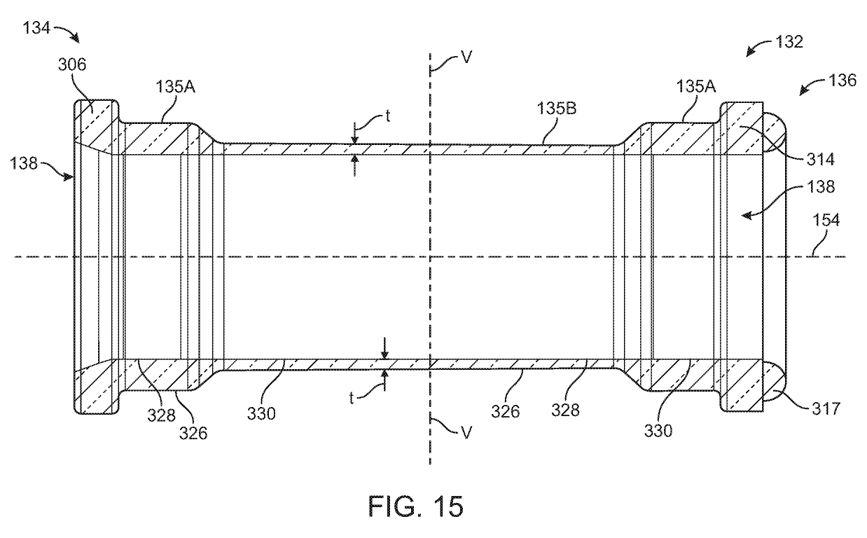

Referring to FIGS. 14 and 15 together, the elongate member 132 can be shaped similarly to the elongate member 132 depicted in FIGS. 12 and 13, but define a narrower wall thickness of wall 330. The wall thickness is identified as reference letter “t”. As noted above, the wall thickness can be between about 0.005 inches and about 0.05 inches, and/or between about 0.010 inches and about 0.030 inches. The thickness can vary along the length of the elongate member 132 between the proximal end 134 and the distal end 136. For example, the thickness can be larger in proximity to the proximal end 134 and the distal end 136 and can be smaller in proximity to the second midsection 135B.

In the illustrated embodiment, the elongate member 132 is a unibody elongate member 132 such that the proximal sealing feature 306, the distal sealing feature 314, and the midsection 135 are fabricated into a unibody construction. In some embodiments, the unibody elongate member 132 can be a single durometer material. In other embodiments, the unibody elongate member 132 can be a multi-durometer material. The durometer can be in the range of about 40A to about 70A. The proximal sealing feature 306 and the distal sealing feature 314 of the elongate member 132 can have a first durometer, and the midsection 135 of the elongate member 132 can have a second durometer. In some examples, the proximal sealing feature 306 and the distal sealing feature 314 of the elongate member 132 can have the first durometer of about 70A, and the midsection 135 of the elongate member 132 can have the second durometer of about 40A. For example, the elongate member 132 can be manufactured from silicone, and the proximal sealing feature 306 and the distal sealing feature 314 of the elongate member 132 can have a durometer of about 70A, and the midsection 135 of the elongate member 132 can have a durometer of about 40A. In some aspects of the present technology, making the durometer of the proximal sealing feature 306 and/or the distal sealing feature 314 greater than a durometer of the midsection 135 allows the midsection 135 to be constricted during operation of the valve 104 while simultaneously ensuring the proximal sealing feature 306 and/or the distal sealing feature 314 generally retain their shape, allowing them to help maintain the position of the unibody elongate member 132 within the housing 128.

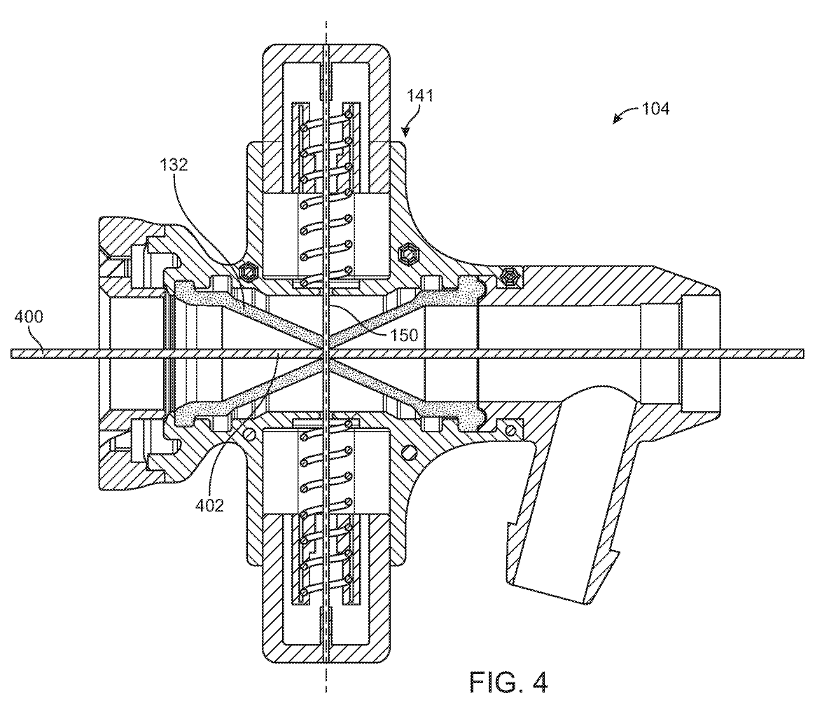

Referring to FIGS. 4 and 14 together, the constricting mechanism 141 can, in some embodiments, collapse and seal the elongate member 132 via compression and/or constriction, and specifically via constriction with at least one filament 150. The constricting mechanism 141 can comprise: an actuator 142, which can be a manual actuator such as one or several buttons 144; and the at least one filament 150 that can extend at least partially around the elongate member 132. In some embodiments, the use of the constricting mechanism 141 can facilitate sealing of the valve around tools or instruments of a wide range of sizes and/or diameters, and particularly around tools or instruments that fit through the elongate member 132.

The housing 128 can further include one or several retention features 140. The one or several retention features 140 of the housing can engage with and retain all or portions of the constricting mechanism 141 of the valve 104. In some embodiments, the one or several retention features 140 of the housing 128 can retain the actuator 142 and/or can couple (e.g., movably couple) the actuator 142 to the housing 128. The actuator 142 can comprise any desired type of actuator including, for example, a manual actuator and/or an automated actuator such as, for example, an electromechanical actuator including a solenoid-based actuator. In some embodiments, the actuator can comprise one or several buttons 144, and specifically, as depicted in FIG. 1, the actuator 142 can comprise a first button 144-A and a second button 144-B that are opposed to one another.

The actuator 142 can be biased toward a configuration, such as, for example, biased toward the first configuration (sealed) or biased toward the second configuration (unsealed). As depicted in FIG. 2, which shows the constricting mechanism 141 in the first configuration, the actuator 142 can be biased toward the first configuration wherein the elongate member 132 is collapsed and/or sealed by a bias feature 146. In this first configuration, the buttons 144 can be in a first position, also referred to herein as an undepressed position. This bias feature 146 can, as shown in FIG. 2, include a first spring 148-A configured to bias the first button 144-A towards the first position corresponding to the first configuration of the constricting mechanism 141, and a second spring 148-B configured to bias the second button 144-B towards a first position corresponding to the first configuration of the constricting mechanism 141. One or both of the first spring 148-A and the second spring 148-B can comprise a tension spring, a compression spring, a torsion spring, a coil spring, or any other desired type of spring.

In some embodiments, one or both of the first spring 148-A and the second spring 148-B can generate sufficient force so as to allow actuation of the actuator 142 with a single hand and so as to collapse and seal the elongate member 132 when the constricting mechanism 141 is in the first configuration. In some embodiments, one or both of the first spring 148-A and the second spring 148-B can generate a force of: at least 0.1 pounds, at least 0.2 pounds, at least 0.3 pounds, at least 0.4 pounds, at least 0.5 pounds, at least 0.6 pounds, at least 0.7 pounds, at least 0.8 pounds, at least 0.9 pounds, at least 1 pound, at least 1.5 pounds, at least 2 pounds, at least 3 pounds, at least 5 pounds, and/or at least 10 pounds and in some embodiments one or both of the first spring 148-A and the second spring 148-B can generate a force approximately between: 0.1 and 10 pounds, 0.1 and 5 pounds, 0.1 and 1.5 pounds, 0.2 and 1 pounds, and/or 0.4 and 0.8 pounds.

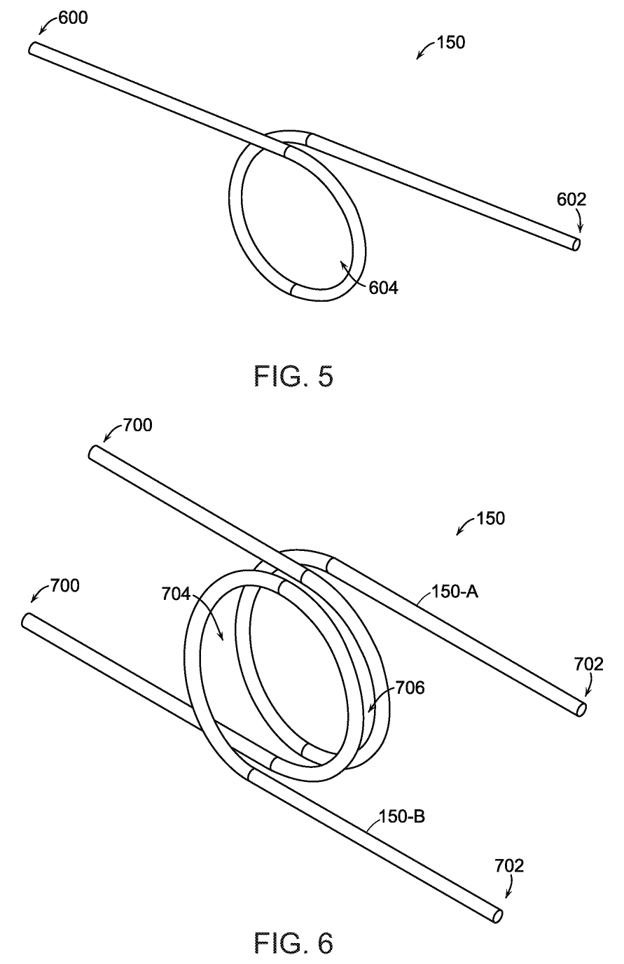

The constricting mechanism 141 can include at least one filament 150 that extends at least partially around the elongate member 132 (See, e.g., FIG. 3). The at least one filament 150 used herein refers generally to the operation of one or more filaments within the valve 104. Different arrangements of the at least one filament (including one or two separate filaments) are illustrated in FIGS. 5-8. In some embodiments, the at least one filament 150 can circumferentially constrict (e.g., when formed in one or more loops) the elongate member 132 to collapse and seal the elongate member 132 when the constricting mechanism 141 is in the first configuration. The filament can be made from a variety of materials, including, for example, a polymer, a synthetic, and/or a metal. In some embodiments, the filament 150 can be nylon, stainless steel, nitinol, silicone, or the like. In some embodiments, the filament can comprise a single strand, such as, for example, a monofilament, and in some embodiments, the filament can comprise a plurality of strands that can be, for example, twisted, woven, grouped, and/or fused to form the filament. In some embodiments, the filament 150 can comprise one or several threads, lines, cords, rope, ribbon, flat wire, sheet, or tape. Regardless of its construction, the filament comprises a thin, flexible length of material formed by one or more strands of material.

The filament 150 can be coupled to the actuator 142 such that the filament 150 selectively constricts, collapses, and/or seals the elongate member 132, and specifically the central lumen 138 of the elongate member 132 based on the movement and/or position of the actuator 142. In some embodiments, the filament 150 can be connected to one or both of the buttons 144-A, 144-B such that the filament 150 collapses, constricts, and/or seals the elongate member 132 and specifically the central lumen 138 of the elongate member 132 when the buttons 144-A, 144-B are in the first position, and the filament 150 can be connected to one or both of the buttons 144-A, 144-B such that the elongate member 132 and specifically the central lumen 138 of the elongate member 132 is open and uncollapsed when the buttons 144-A, 144-B are in the second position. In some embodiments in which the actuator 142 comprises a single button 144 (not shown), the filament 150 can be connected to the button 144 and to the housing 128 such that the filament 150 is tightened when the button 144 moves to the first position.

In some embodiments, the at least one filament 150 can extend along an axis 152 that can be perpendicular to the central axis 154 of the elongate member 132 and/or of the apertures 124, 126. In some embodiments, the axis 152 of the at least one filament 150 can intersect and be perpendicular to the central axis 154 of the elongate member 132 and/or of the apertures 124, 126. In some embodiments, the actuator 142, and specifically the buttons 144-A, 144-B can move along this axis 152 when moved from the first position to the second position.

Referring to FIG. 3, an embodiment of the valve 104 with the constricting mechanism 141 in the second configuration is shown. As specifically shown, both of the first and second buttons 144-A, 144-B are in the second position, depressed into the retention features 140 of the housing 128. In this second position, the filament 150 is loosened, thereby allowing the expansion of the elongate member 132 and the unsealing of the central lumen 138 of the elongate member 132.

Referring to FIG. 3, the proximal cap 116 has a proximal end 300 and a distal end 302. The proximal cap 116 can include a funnel portion 301 of the proximal aperture 124, which funnel portion 301 can facilitate insertion of a tool into the proximal aperture 124. The distal end 302 of the proximal cap 116 can partially extend into the interior channel 130 of the housing 128. The proximal cap 116 can include a mating feature 304 that can mate with the proximal end 134 of the elongate member 132. In some embodiments, the proximal end 134 of the elongate member 132 can fit over the mating feature 304 of the proximal cap 116. The proximal end 134 of the elongate member 132 can be compressed between the mating feature 304 of the elongate member 132 and a portion of the interior channel 130 of the housing 128 into which the mating feature 304 is inserted to thereby secure the proximal end 134 of the elongate member 132 on the mating feature 304. The proximal end 134 of the elongate member 132 is secured to the mating feature 304 by the proximal sealing feature 306 that can be compressed between the housing 128 and the mating feature 304 of the proximal cap 116 to sealingly couple the elongate member 132 to the proximal cap 116. In the illustrated embodiment, the elongate member 132 includes the proximal sealing feature 306 as part of the unibody construction.

In some embodiments, the proximal end 134 of the elongate member 132 is secured to the mating feature 304 by an adhesive. In some instances, the proximal end 134 of the elongate member 132 can be secured to the mating feature 304 by a proximal sealing feature 306, as described above, and by an adhesive. In some embodiments, the proximal cap 116 is secured to the mating feature 304 by an adhesive.

The distal cap 120 has a proximal end 308 and a distal end 310. The distal cap can include a mating feature 312 located on the proximal end 308 of the distal cap 120, which mating feature 312 can mate with the distal end 136 of the elongate member 132. In some embodiments, the distal end 136 of the elongate member 132 can fit over the mating feature 312 of the distal cap 120. The distal end 136 of the elongate member 132 can be compressed between the mating feature 312 of the elongate member 132 and a portion of the interior channel 130 of the housing 128 into which the mating feature 312 is inserted to thereby secure the distal end 136 of the elongate member 132 on the mating feature 312. In some embodiments, the distal end 136 of the elongate member 132 is secured to the mating feature 312 by the distal sealing feature 314 that can be compressed between the housing 128 and the mating feature 312 of the proximal cap 116 to sealingly couple the elongate member 132 to the distal cap 120. In the illustrated embodiment, the elongate member 132 includes the distal sealing feature 314 as part of the unibody construction.

In a non-limiting example, the benefit of the elongate member 132 having a unibody construction is, among other benefits, fewer components and fewer assembly steps. In addition, the unibody construction allows the elongate member 132 to be manufactured into a desired shape with a desired material(s).

The distal cap 120 can, in some embodiments, further include a side port barb 315 that can extend laterally away from the distal cap 120 and specifically away from the distal aperture 126 of the distal cap 120. The side port barb 315 can define a side port channel 316 that can extend through the side port barb 315 and fluidly connect to the distal aperture 126. In some embodiments, the side port barb 315 can include a securement feature 318, such as a barb that can secure coupling of a hose or tube to the side port barb 315.

In some embodiments, the side port barb 315 can be used to apply a vacuum to the portions of the delivery device 100, and particularly to portions of the delivery device 100 that are distal of the axis 152 along which the elongate member 132 seals. This vacuum can be applied to aspirate a material through the delivery device 100, and specifically through the catheter 102 of the delivery device. This aspirated material can be a biological material including, for example, bodily fluids, multi-phase bodily materials that can include, for example, a fluidic portion and at least one solid portion, or the like.

In some embodiments, due to the narrowing shape of the elongate member 132 when the constricting mechanism 141 is in the first configuration, a vacuum applied to the portions of the delivery device 100 distal to the axis 152 draws the elongate member 132 towards the first configuration and can, in some embodiments, increase the strength, robustness, and/or strength of the seal of the valve 104. This attribute of the valve 104 can provide benefits over other valve designs in which a vacuum can compromise the seal of the valve, and thus the ability to draw a vacuum and aspirate can be limited.

In some embodiments, the valve 104 can further include a reinforcement structure 320 that can extend along all or portions of the elongate member 132. The reinforcement structure 320 can facilitate the uniform collapse of the elongate member 132, can inhibit, or even prevent, the at least one filament 150 from cutting through and/or tearing the elongate member 132, and can assist in guiding one or several tools through the elongate member 132. The reinforcement structure 320 can be tubular, can extend along and around the elongate member 132, and can be positioned so as to be between the elongate member 132 and the at least one filament 150.

The reinforcement structure 320 can include a proximal end 322 and a distal end 324. In some embodiments, the reinforcement structure 320 extends along and around the elongate member 132, and is positioned such that the proximal end 322 of the reinforcement structure 320 is proximate to the first end 134 of the elongate member 132 and the distal end 324 of the reinforcement structure 320 is proximate to the second end 136 of the elongate member 132.

The reinforcement structure 320 can be coupled to the elongate member 132. In some embodiments, the reinforcement structure 320 is coupled to the elongate member 132 along the length of the reinforcement structure 320, and in some embodiments, the reinforcement structure 320 is coupled to the elongate member 132 and distinct positions along the length of the elongate member 132 and/or the reinforcement structure 320. In one embodiment, for example, the reinforcement structure 320 can be coupled to the elongate member 132 at one or both of the proximal end 322 of the reinforcement structure 320 and the distal end 324 of the reinforcement structure 320 and/or at one or both of the first end 134 and the second end 136 of the elongate member 132. In some embodiments, the reinforcement structure 320 can be coupled to the elongate member 132 via one or several other components of the valve 104. In some embodiments, the reinforcement structure 320 can be coupled to the elongate member 132 via the compression of the reinforcement structure 320 and the elongate member 132 between the housing 128 and one or both of the proximal cap 116 and the distal cap 120.

In some embodiments, the reinforcement structure 320 can be adhered to the elongate member 132 via, for example, an adhesive such as silicone adhesive. In some embodiments, the adhesive can be circumferentially applied to the reinforcement structure 320 and/or the elongate member 132 in an adhesive ring that can, for example, have a length approximately between: 0.010 inches and 0.5 inches; 0.02 and 0.4 inches; 0.050 inches and 0.0250 inches, or any other or intermediate range.

In one embodiment, each of the proximal end 322 and the distal end 324 of the reinforcement structure 320 can be adhered via an adhesive to the elongate member 132. In such an embodiment, the reinforcement structure 320 can be uncoupled to the elongate member 132 at positions other than the coupling at one or both of the proximal end 322 and the distal end 324 of the reinforcement structure 320, and thus the reinforcement structure 320 is uncoupled to the elongate member 132 at a position between the first end 134 and the second end 136 of the elongate member 132 and/or between the proximal end 322 and the distal end 324 of the reinforcement structure 320.

Referring to FIGS. 3 and 4 together, the lack of coupling of the reinforcement structure 320 to the elongate member 132 can facilitate and improve the collapse of the elongate member 132 around a tool 400, also referred to herein as instrument 400 or device 400, inserted through the valve 104. The tool 400 can be any device inserted through the valve 104, including, for example, one or several additional catheters, lines, wires, grippers, punches, cutters, or the like. As shown in FIG. 4, the tool 400 is inserted through the valve 104 and specifically through the elongate member 132 of the valve. As shown, the constricting mechanism 141 is in the first configuration, and the elongate member 132 and the central lumen 138 of the elongate member 132 are collapsed around the tool 400, and specifically around a shaft 402 of the tool 400, to thereby seal the valve 104 around the tool 400 and specifically around the shaft 402 of the tool 400. The constricting mechanism 141 can seal around tools 400 that fit through the elongate member 132, regardless of the size of the tool 400. Thus, the valve can be used with a wide variety of tools.

The reinforcement structure 320 can include a variety of designs, shapes, sizes, and materials. In some embodiments, the reinforcement structure 320 can be sized and shaped so as to receive elongate member 132 and to be positioned between the elongate member 132 and the at least one filament 150. In some embodiments, the reinforcement structure 320 can be made from a material sufficiently strong to inhibit, or even prevent, the cutting of the at least one filament 150 through the elongate member 132. In some embodiments, the reinforcement structure can comprise a coil or a mesh sheath. The mesh sheath can, in some embodiments, comprise a braided mesh. The braided mesh can be made from any desired number of wires in any desired configuration. In some embodiments, the braided mesh can comprise a 4 wire braided mesh, an 8 wire braided mesh, a 12 wire braided mesh, a 16 wire braided mesh, a 20 wire braided mesh, a 24 wire braided mesh, a 32 wire braided mesh, a 48 wire braided mesh, a 64 wire braided mesh, a 72 wire braided mesh, an 80 wire braided mesh, a 96 wire braided mesh, or any other or intermediate braided mesh. In some embodiments, the braided mesh can comprise: a 1×1 configuration. In some embodiments, the wire in the braided mesh can be any desired material, including, for example, a metal wire such as a nitinol wire or a stainless steel wire, a polymer wire, or a natural wire. In one embodiment, the braided mesh can comprise a 48 wire mesh in a 1×1 configuration made with a nitinol wire having a diameter of 0.003 inches.

With reference now to FIGS. 5 through 8, different embodiments and/or configurations of the at least one filament 150 are shown. The filament 150 can comprise a single filament 150 having a first end 600 and a second end 602 as shown in FIG. 5. The filament 150, and specifically which first and second ends 600, 602 can be coupled to the actuator 142 to move the filament 150 between the first and second configurations or positions and/or from the first configuration or position to the second configuration or position. In some embodiments, both of the first end 600 and the second end 602 can be coupled to a single button 144, in some embodiments, each of the first end 600 and the second end 602 can be coupled to different buttons 144, and in some embodiments, one of the first end 600 and the second end 602 can be coupled to a button 144 and the other of the first end 600 and the second end 602 can be coupled to the housing 128 or other portion of the valve 104.

In some embodiments, the filament 150 can comprise multiple filaments, and specifically, as shown in FIGS. 6 through 8, the filament 150 can comprise a first filament 150-A and a second filament 150-B. In embodiments in which the filament 150 comprises multiple filaments, each of the multiple filaments can have a first end 700 and a second end 702. The first and second filaments 150-A, 150-B can be coupled to the actuator 142. In such embodiments, the first and second ends 700, 702 can be coupled to the actuator 142 to move the first and second filaments 150-A, 150-B between the first and second configurations and/or from the first configuration to the second configuration. In some embodiments, both of the first end 700 and the second end 702 of one or more of the multiple filaments 150 can be coupled to a single button 144, in some embodiments, each of the first end 700 and the second end 702 of one or more of the multiple filaments 150 can be coupled to different buttons 144, and in some embodiments, one of the first end 700 and the second end 702 of one or more of the multiple filaments 150 can be coupled to one button 144 and the other of the first end 700 and the second end 702 of those one or more filaments 150 can be coupled to the housing 128 or other portion of the valve 104.

The filament 150 can be arranged in a variety of configurations. In some embodiments, the filament 150 comprises one filament and is positioned to form a single loop 604 that can extend around the elongate member 132 and/or through which the elongate member 132 can be received as shown in FIG. 5, and in some embodiments, the filament 150 can comprise multiple filaments each positioned to form a loop, and specifically a first loop 704 and a second loop 706 as shown in FIG. 6. The first and second loops 704, 706 can each receive the elongate member 132. In some embodiments, a diameter or size of the loop 604, or of the loops 704, 706 can decrease when the constricting mechanism 141 is moved from the second configuration to the first configuration.

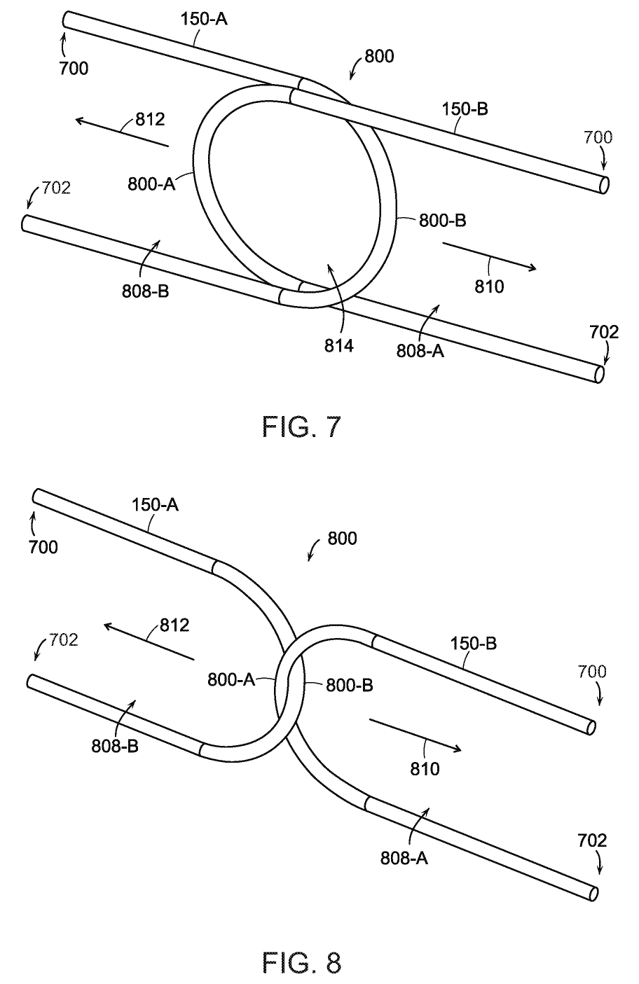

In some embodiments, the filament 150 can comprise one or multiple filaments (e.g., two filaments) each configured to form a bight. As used herein, a “bight” refers to a U-shaped section between the two ends of the filament 150. As depicted in FIGS. 7 and 8, a bight 800 can comprise multiple bights, and specifically a first bight 800-A formed by the first filament 150-A and a separate second bight 800-B formed by the separate second filaments 150-B. In some embodiments, the first bight 800-A can extend through the second bight 800-B such that the first and second bights 800-A, 800-B interlock, whereas in other embodiments, the first and second bights 800-A, 800-B can be non-interlocking. Similarly, in embodiments containing the filament 150 having multiple loops, one or several of the multiple loops can be interlocking.

In some embodiments, the bight 800, and specifically one or both of the first bight 800-A and the second bight 800-B can be formed around a portion of the elongate member 132 and/or can extend around a portion of the elongate member 132. Each bight 800 can define a partially enclosed receiving area 808 wherein the elongate member 132 can be received. Thus, the first bight 800-A can define a first receiving area 808-A, and the second bight 800-B can define a second receiving area 808-B.

Referring to FIGS. 7 and 8 together, multiple bights, and specifically the first and second bights 800-A, 800-B can be positioned and oriented such that the first bight 800-A has a first orientation or first direction as indicated by arrow 810, and the second bight has a second orientation or second direction as indicated by the arrow 812. In some embodiments, the first orientation is different from the second orientation such that the first and second receiving areas 808-A, 808-B overlap and define an encircled area 814, also referred to herein as a constricting area 814. The elongate member 132 can be received within the encircled area 814. In embodiments in which bights 800-A, 800-B overlap to define the encircled area 814, the movement of the constricting mechanism 141 to the first configuration can result in and/or include the first bight 800-A moving in the direction indicated by the arrow 810 and/or the second bight 800-B moving in the direction indicated by the arrow 812, which movement of the bights 800-A, 800-B decreases the size of the encircled area 814 and constricts, collapses, and/or seals the elongate member 132 extending through the encircled area 814.

The filament(s) 150 forming the bights 800 can each apply an arcuate line or narrow longitudinal zone of pressure to the elongate member 132. If the filament(s) are circular in cross-section, the zone of pressure can be very small, and can, in some embodiments, be less than the diameter or thickness of the filament. In some embodiments, the filaments have a diameter or width less than about 2.5 mm, less than about 2 mm, less than about 1.5 mm, less than about 1.25 mm, less than about 1 mm, less than about 0.75 mm, less than about 0.5 mm, and/or less than about 0.25 mm. In some embodiments, the filaments can have a diameter or width of between about 0.01 mm and 2.5 mm, between about 0.05 mm and 2 mm, between about 0.1 mm and 1 mm, and/or between about 0.125 mm and 0.70 mm. In some embodiments, the arcuate line or zone of pressure can form two opposing arcs, and in other embodiments, the arcuate line of pressure can be a singular, substantially circular line or zone that encircles the elongate member at least once. The longitudinal length of the line or zone of pressure can be very short compared to other valves known in the art. In some embodiments, the longitudinal length of the zone of pressure applied to the elongate member 132 by the filament(s) 150 can be less than about 2.0 mm and in some embodiments less than about 0.5 mm. In some embodiments, the filament(s) 150 can have any desired cross-sectional shape, including, for example, a circular cross-section, a rectangular cross-section, an oval cross-section, a square cross-section, a polygonal cross-section, a triangular cross-section, or any other desired shape of cross-section.

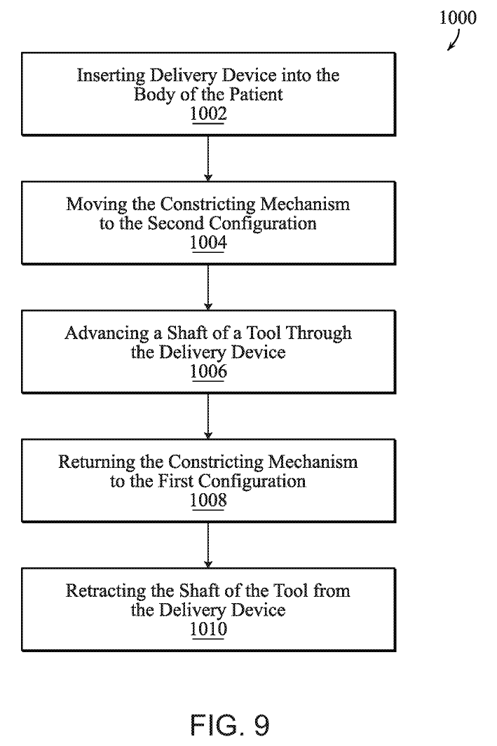

Referring to FIG. 9, a flowchart illustrating one embodiment of a process 1000 for sealing a valve 104 and/or catheter 102 accessing a body of a patient is shown. The process 1000 can be performed using the valve 104 and/or the delivery system 100. The process 1000 begins at block 1002, wherein the delivery device 100, and specifically the catheter 102 of the delivery device 100 is inserted into the body of the patient. In some embodiments, this can include inserting the catheter 102 into a portion of the circulator system of the patient, such as, for example, a blood vessel, including an artery or a venous vessel. In some embodiments, the delivery device 100 can be inserted into the body of the patient directly through an aperture or incision in the patient, and in some embodiments, the delivery device 100 can be inserted into the body of the patient via another catheter or device. In some embodiments, the constricting mechanism 141 can be in the first configuration while the delivery device 100 and/or the catheter 102 is inserted into the patient's body.

After the delivery device 100 is inserted into the body of the patient, the process 1000 proceeds to block 1004, wherein the constricting mechanism 141 is moved from the first configuration to the second configuration. As described above, the central lumen 138 of the unibody elongate member 132 is unsealed when the constricting mechanism 141 is in the second configuration. In some embodiments, the moving of the constricting mechanism 141 from the first configuration to the second configuration can include the manipulation of the actuator 142 and/or the control of the actuator 142, and specifically the depressing of the one or several buttons 144 to move the filament 150 from the first position to the second position to allow the expansion and opening of the central lumen 138 of the elongate member 132.

After the constricting mechanism 141 is moved from the first configuration to the second configuration, the process 1000 proceeds to block 1006, wherein the tool 400, and specifically the shaft 402 of the tool 400 is advanced through the delivery device 100 and specifically through the valve 104 until a first end of the tool reaches a desired position within the body of the patient. In some embodiments, a portion of the shaft 402 can be positioned within the central lumen 138 of the elongate member 132 after the advancement of the tool 400 through the delivery device 100. In some embodiments, after the tool 400 is advanced through the delivery device 100, the desired procedure can be performed with the tool.

After the tool 400 is advanced through the delivery device 100, or while the tool 400 is being advanced through the delivery device 100, the process 1000 proceeds to block 1008, wherein the constricting mechanism 141 is returned to the first configuration. In some embodiments, the returning of the constricting mechanism 141 to the first configuration can include the release of the one or several buttons 144 and/or the control of the actuator 142 to reconfigure the constricting mechanism 141 to the first configuration. In some embodiments, the return of the constricting mechanism 141 to the first configuration can result in the collapse and/or sealing of the elongate member 132 and specifically the central lumen 138 of the elongate member 132 around the tool 400 and specifically around the shaft 402 of the tool 400. The return of the constricting mechanism 141 to the first configuration, or the movement of the constricting mechanism 141 to the first configuration can include the decreasing of the size and/or diameter of one or several loops formed by the filament 150 and/or the movement of one or several bights 800 such as, for example, the movement of the first bight 800-A in the first direction indicated by arrow 810 and the movement of the second bight 800-B in the second direction indicated by arrow 812 to reduce the size of the constricting area 814. In some embodiments, after the constricting mechanism is returned to the first configuration, the desired procedure can be performed with the tool.

After the constricting mechanism is returned to the first configuration, the process 1000 proceeds to block 1010, wherein the tool 400, and specifically the shaft 402 of the tool 400 is retracted from the delivery device 100, and more specifically from the valve 104. In some embodiments, the valve 104 can remain sealed during the retracing of the tool 400 and/or the shaft 402 of the tool. In some embodiments, the valve 104 remains sealed during the retracting of the tool 400 and/or the retracting of the shaft 402 of the tool 400 as the constricting mechanism 141 can remain in the first configuration during the retracing of the tool 400 and/or the shaft 402 of the tool 400.

In some embodiments, the constricting mechanism 141 can be moved to the second configuration to allow the retraction of the tool 400 and/or the shaft 402 of the tool 400 from the valve 104, and the constricting mechanism 141 can be returned to the first configuration when the tool 400 and/or the shaft 402 of the tool 400 is removed from the valve 104. In some embodiments, the retraction of the tool 400 and/or shaft 402 of the tool 400 from the valve 104 can be performed with the constricting mechanism 141 left in the first configuration. In some embodiments, the constricting mechanism 141 can be moved to the second configuration, and then returned to the first configuration via the manipulation and/or control of the actuator 142, which manipulation and/or control of the actuator 142 can include the depressing of the one or several buttons 144 to move the constricting mechanism 141 to the second configuration, and the release of the one or several buttons 144 to return the constricting mechanism 141 to the first configuration. In some embodiments, if the procedure is complete, the delivery device 100 can then be removed from the body of the patient, and any incision created for the procedure can be closed.

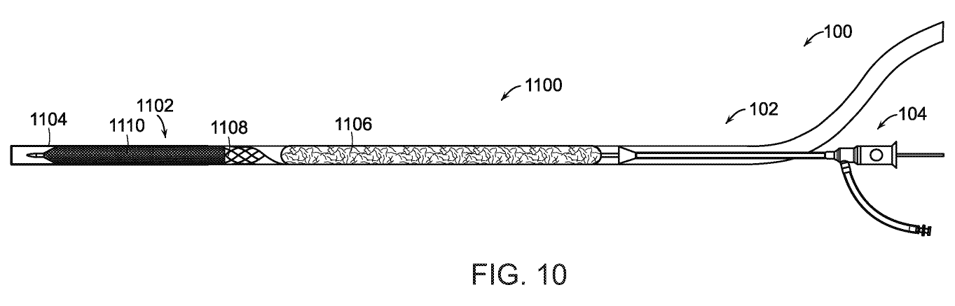

Referring to FIG. 10, a side view of one embodiment of a thrombectomy system 1100 including the delivery device 100 and a thrombus extraction device 1102 is shown. In some embodiments, the thrombectomy system 1100 can be used to access a blood vessel 1104 to treat and/or extract a thrombus 1106 from the blood vessel 1104. The thrombus extraction device 1102 can include a self-expanding coring element 206 and an expandable cylindrical portion 208. In some embodiments, and as shown in FIG. 10, the thrombus extraction device 1102 can be the tool 400 that can extend through the valve 104, and in some embodiments, the valve 104 can be a part of the thrombus extraction device 1102. Further details of thrombectomy systems, thrombus extraction devices, and methods of using the same are disclosed in: (i) U.S. Patent Application No. 15/268,296, filed September 16, 2016, and titled “INTRAVASCULAR TREATMENT OF VASCULAR OCCLUSION AND ASSOCIATED DEVICES, SYSTEMS, AND METHODS”, now Patent No. 9,700,332; (ii) U.S. Patent Application No. 15/498,320, filed Apr. 26, 2017, and titled “DEVICES AND METHODS FOR TREATING VASCULAR OCCLUSION”, now Patent No. 10,098,651; (iii) U.S. Patent Application No. 15/466,740, filed on Mar. 22, 2017, and titled “DEVICE AND METHOD FOR TREATING VASCULAR OCCLUSION”, now Patent No. 10,045,790; (iv) U.S. Patent Application No. 16/536,185, filed Aug. 8, 2019, and titled “SYSTEM FOR TREATING EMBOLISM AND ASSOCIATED DEVICES AND METHODS”, now Patent No. 11,559,382; and (v) U.S. Patent Application No. 16/258,344, filed Jan. 25, 2019, and titled “SINGLE INSERTION DELIVERY SYSTEM FOR TREATING EMBOLISM AND ASSOCIATED SYSTEMS AND METHODS”, now Patent No. 11,154,314, the entirety of each of which is hereby incorporated by reference herein.

### III. Additional Examples

Several aspects of the present technology are set forth in the following examples:

1. A hemostatic valve for sealing a medical device, the hemostatic valve comprising:

a unibody elongate member having a first end, a second end, and a central lumen extending therebetween, wherein the unibody elongate member is pliable;

an active tensioning mechanism including an actuator coupled to the unibody elongate member, wherein the actuator is moveable between (a) a first position wherein the central lumen is constricted and sealed and (b) a second position wherein the central lumen is open, and wherein the tensioning mechanism comprises at least one filament extending at least partially around the unibody elongate member; and

a biasing member configured to bias the actuator to the first position.

2. The hemostatic valve of example 1, wherein the unibody elongate member comprises a proximal sealing feature positioned at the first end and a distal sealing feature positioned at the second end opposite from the first end.

3. The hemostatic valve of example 2, wherein the unibody elongate member comprises a midsection situated between the proximal sealing feature and the distal sealing feature.

4. The hemostatic valve of any of examples 1–3 further comprising a reinforcement structure extending along at least a portion of the unibody elongate member, wherein the reinforcement structure is coupled to the unibody elongate member.

5. The hemostatic valve of example 4, wherein the reinforcement structure is positioned between the at least one filament and the unibody elongate member.

6. The hemostatic valve of example 5, wherein the reinforcement structure comprises a braided mesh.

7. The hemostatic valve of example 4, wherein the reinforcement structure is coupled to the unibody elongate member at a position proximate to the first end of the unibody elongate member and at a position proximate to the second end of the unibody elongate member.

8. The hemostatic valve of any of examples 1–7, wherein when the actuator is in the first position, the central lumen is configured to remain constricted and sealed when a pressure differential exists between (a) a first volume outside the central lumen and adjacent to the first end of the unibody elongate member and (b) a second volume outside the central lumen and adjacent to the second end of the unibody elongate member.

9. The hemostatic valve of any of examples 1–8, wherein when the actuator is in the first position, the central lumen is configured to remain constricted and sealed when vacuum pressure is applied to a volume outside the central lumen and adjacent to either the first end or the second end of the unibody elongate member.

10. The hemostatic valve of any of examples 1–9, wherein the unibody elongate member is a multi-durometer elongate member.

11. The hemostatic valve of example 10, wherein the durometer is in the range of about 40A to about 70A.

12. The hemostatic valve of example 3, wherein the unibody elongate member is a multi-durometer elongate member, wherein the proximal and distal sealing features have a first durometer and the midsection has a second durometer.

13. The hemostatic valve of example 12, wherein the first durometer is in the range of about 40A to about 70A, and the second durometer is in the range of about 40A to about 70A.

14. The hemostatic valve of example 13, wherein the first durometer is about 70A and the second durometer is about 40A.

15. The hemostatic valve of example 12, wherein the first durometer is higher than the second durometer.

16. The hemostatic valve of example 12, wherein the first durometer and the second durometer are different.

17. The hemostatic valve of any of examples 1–16, wherein the unibody elongate member defines a midsection extending between the first end and the second end, wherein the midsection defines a straight tapered cross-section.

18. The hemostatic valve of example 17, wherein the midsection tapers a first distance from the first end in the direction of the second end at a first angle, and the midsection tapers a second distance from the second end in the direction of the first end at a second angle.

19. The hemostatic valve of any of examples 1–18, wherein the unibody elongate member defines a midsection extending between the first end and the second end, wherein the midsection defines a step-tapered cross-section.

20. The hemostatic valve of example 19, wherein the midsection defines a first midsection in proximity to the first end, wherein the first midsection transitions to a second midsection in the direction of the second end, and the midsection defines the first midsection in proximity to the second end, wherein the first midsection transitions to the second midsection in the direction of the first end.

21. The hemostatic valve of example 20, wherein the first midsection defines a width that is greater than the second midsection.

22. The hemostatic valve of any of examples 1–21, wherein the unibody elongate member defines a thickness between about 0.005 inches and 0.05 inches.

23. A hemostatic valve for sealing a medical device, the hemostatic valve comprising:

a unibody elongate member having a first end, a second end, and a central lumen extending therebetween, wherein the unibody elongate member is pliable;

an active tensioning mechanism including an actuator coupled to the unibody elongate member and at least one filament coupled to the actuator and extending at least partially around the unibody elongate member, wherein the actuator is moveable between (a) a first position wherein the central lumen is constricted and sealed and (b) a second position wherein the central lumen is open, and wherein, in the first position, the actuator pulls the at least one filament to collapse the unibody elongate member such that the central lumen is constricted and sealed; and

a biasing member configured to bias the actuator to the first position.

24. The hemostatic valve of example 23, wherein the unibody elongate member is a multi-durometer elongate member.

25. The hemostatic valve of example 24, wherein the durometer is in the range of about 40A to about 70A.

26. The hemostatic valve of any of examples 23–25, wherein the unibody elongate member defines a midsection extending between the first end and the second end, wherein the midsection defines a straight tapered cross-section.

27. The hemostatic valve of example 26, wherein the midsection tapers a first distance from the first end in the direction of the second end at a first angle, and the midsection tapers a second distance from the second end in the direction of the first end at a second angle.

28. The hemostatic valve of any of examples 23–27, wherein the unibody elongate member defines a midsection extending between the first end and the second end, wherein the midsection defines a step-tapered cross-section.

29. The hemostatic valve of example 28, wherein the midsection defines a first midsection in proximity to the first end, wherein the first midsection transitions to a second midsection in the direction of the second end, and the midsection defines the first midsection in proximity to the second end, wherein the first midsection transitions to the second midsection in the direction of the first end.

30. The hemostatic valve of example 29, wherein the first midsection defines a width that is greater than the second midsection.

31. The hemostatic valve of any of examples 23–30, wherein the unibody elongate member comprises a proximal sealing feature positioned at the first end and a distal sealing feature positioned at the second end opposite from the first end.

32. The hemostatic valve of example 31, wherein the unibody elongate member comprises a midsection situated between the proximal sealing feature and the distal sealing feature.

33. The hemostatic valve of example 32, wherein the unibody elongate member is a multi-durometer elongate member, wherein the proximal and distal sealing features have a first durometer and the midsection has a second durometer.

34. The hemostatic valve of example 33, wherein the first durometer is in the range of about 40A to about 70A, and the second durometer is in the range of about 40A to about 70A.

35. The hemostatic valve of example 34, wherein the first durometer is about 70A and the second durometer is about 40A.

36. The hemostatic valve of example 33, wherein the first durometer is higher than the second durometer.

37. The hemostatic valve of example 33, wherein the first durometer and the second durometer are different.

38. The hemostatic valve of any of examples 23–37, wherein the unibody elongate member defines a thickness between about 0.005 inches and 0.05 inches.

39. A unibody elongate member configured for use within a hemostatic valve, the unibody elongate member comprising:

a proximal sealing feature positioned at a first end of the unibody elongate member;

a distal sealing feature positioned at a second end of the unibody elongate member opposite from the first end; and

a midsection positioned between the proximal sealing feature and the distal sealing feature,

wherein the proximal sealing feature defines a first cross-sectional width, the distal sealing feature defines a second cross-sectional width, and the midsection defines a third cross-sectional width that is smaller than the first and second cross-sectional widths.

40. The unibody elongate member of example 39, wherein the unibody elongate member is a multi-durometer elongate member, wherein the proximal and distal sealing features have a first durometer and the midsection has a second durometer.

41. The hemostatic valve of example 40, wherein the first durometer is in the range of about 40A to about 70A, and the second durometer is in the range of about 40A to about 70A.

42. The hemostatic valve of example 41, wherein the first durometer is about 70A and the second durometer is about 40A.

43. The hemostatic valve of example 40, wherein the first durometer is higher than the second durometer.

44. The hemostatic valve of example 40, wherein the first durometer and the second durometer are different.

45. The hemostatic valve of any of examples 39–44, wherein the midsection defines a straight tapered cross-section.

46. The hemostatic valve of example 45, wherein the midsection tapers a first distance from the first end in the direction of the second end at a first angle, and the midsection tapers a second distance from the second end in the direction of the first end at a second angle.

47. The hemostatic valve of any of examples 39–46, wherein the midsection defines a step-tapered cross-section.

48. The hemostatic valve of example 47, wherein the midsection defines a first midsection in proximity to the first end, wherein the first midsection transitions to a second midsection in the direction of the second end, and the midsection defines the first midsection in proximity to the second end, wherein the first midsection transitions to the second midsection in the direction of the first end.

49. The hemostatic valve of example 48, wherein the first midsection defines a width that is greater than the second midsection.

50. The hemostatic valve of any of examples 39–49, wherein the unibody elongate member defines a thickness between about 0.005 inches and 0.05 inches.

51. A hemostatic valve for sealing a medical device, the hemostatic valve comprising:

a unibody elongate member having a proximal sealing feature, a distal sealing feature, and a central lumen extending therebetween, wherein the unibody elongate member is pliable;

a tensioning mechanism including an actuator coupled to the unibody elongate member, wherein the actuator is moveable between (a) a first position wherein the central lumen is constricted and sealed and (b) a second position wherein the central lumen is open, and wherein the tensioning mechanism comprises at least one filament extending at least partially around the unibody elongate member; and

a biasing member configured to bias the actuator to the first position.

52. The hemostatic valve of example 51, wherein the proximal sealing feature is positioned at a first end of the unibody elongate member, and the distal sealing feature is positioned at a second end opposite from the first end.

53. The hemostatic valve of either example 51 or example 52, wherein the unibody elongate member comprises a midsection situated between the proximal sealing feature and the distal sealing feature.

54. The hemostatic valve of any of examples 51–53 further comprising a reinforcement structure extending along at least a portion of the unibody elongate member, wherein the reinforcement structure is coupled to the unibody elongate member.

55. The hemostatic valve of example 54, wherein the reinforcement structure is positioned between the at least one filament and the unibody elongate member.

56. The hemostatic valve of example 55, wherein the reinforcement structure comprises a braided mesh.

57. The hemostatic valve of example 54, wherein the reinforcement structure is coupled to the unibody elongate member at a position proximate to the proximal sealing feature and at a position proximate to the distal sealing feature.

58. The hemostatic valve of any of examples 51–57, wherein when the actuator is in the first position, the central lumen is configured to remain constricted and sealed when a pressure differential exists between (a) a first volume outside the central lumen and adjacent to the proximal sealing feature and (b) a second volume outside the central lumen and adjacent to the distal sealing feature.

59. The hemostatic valve of any of examples 51–58, wherein when the actuator is in the first position, the central lumen is configured to remain constricted and sealed when vacuum pressure is applied to a volume outside the central lumen and adjacent to either the proximal sealing feature or the distal sealing feature.

60. The hemostatic valve of any of examples 51–59, wherein the unibody elongate member is a multi-durometer elongate member.

61. The hemostatic valve of example 60, wherein a durometer of the multi-durometer elongate member is in a range of about 40A to about 70A.

62. The hemostatic valve of example 53, wherein the unibody elongate member is a multi-durometer elongate member, wherein the proximal and distal sealing features have a first durometer, and the midsection has a second durometer.

63. The hemostatic valve of example 62, wherein the first durometer is in a range of about 40A to about 70A, and the second durometer is in the range of about 40A to about 70A.

64. The hemostatic valve of example 63, wherein the first durometer is about 70A and the second durometer is about 40A.

65. The hemostatic valve of example 62, wherein the first durometer is greater than the second durometer.

66. The hemostatic valve of example 62, wherein the first durometer and the second durometer are different.

67. The hemostatic valve of any of examples 51–66, wherein the unibody elongate member includes a midsection extending between the proximal sealing feature and the distal sealing feature, wherein the midsection defines a straight tapered cross-section.

68. The hemostatic valve of example 67, wherein the midsection tapers a first distance from the proximal sealing feature in a first direction at a first angle, and the midsection tapers a second distance from the distal sealing feature in a second direction at a second angle.

69. The hemostatic valve of any of examples 51–68, wherein the unibody elongate member includes a midsection extending between the proximal and distal sealing features, wherein the midsection defines a step-tapered cross-section.

70. The hemostatic valve of example 69, wherein the midsection defines a first midsection in proximity to the proximal sealing feature, wherein the first midsection transitions to a second midsection in a first direction, and the midsection defines the first midsection in proximity to the distal sealing feature, wherein the first midsection transitions to the second midsection in a second direction.

71. The hemostatic valve of example 70, wherein the first midsection defines a width that is greater than the second midsection.

72. The hemostatic valve of any of examples 51–71, wherein the unibody elongate member defines a thickness between about 0.005 inches and 0.05 inches.

73. A hemostatic valve for sealing a medical device, the hemostatic valve comprising:

a unibody elongate member having a first sealing feature, a second sealing feature, and a central lumen extending therebetween, wherein the unibody elongate member is pliable;

an active tensioning mechanism including an actuator coupled to the unibody elongate member and at least one filament coupled to the actuator and extending at least partially around the unibody elongate member, wherein the actuator is moveable between (a) a first position wherein the central lumen is constricted and sealed and (b) a second position wherein the central lumen is open, and wherein, in the first position, the actuator pulls the at least one filament to collapse the unibody elongate member such that the central lumen is constricted and sealed; and

a biasing member configured to bias the actuator to the first position.

74. The hemostatic valve of example 73, wherein the unibody elongate member is a multi-durometer elongate member.

75. The hemostatic valve of example 74, wherein a durometer of the multi-durometer elongate member is in a range of about 40A to about 70A.

76. The hemostatic valve of any of examples 73–75, wherein the unibody elongate member includes a midsection extending between the first sealing feature and the second sealing feature, wherein the midsection defines a straight tapered cross-section.

77. The hemostatic valve of example 76, wherein the midsection tapers a first distance from the first sealing feature in a first direction at a first angle, and the midsection tapers a second distance from the second sealing feature in a second direction at a second angle.

78. The hemostatic valve of any of examples 73–77, wherein the unibody elongate member includes a midsection extending between the first sealing feature and the second sealing feature, wherein the midsection defines a step-tapered cross-section.

79. The hemostatic valve of example 78, wherein the midsection defines a first midsection in proximity to the first sealing feature, wherein the first midsection transitions to a second midsection in a first direction, and the midsection defines the first midsection in proximity to the second sealing feature, wherein the first midsection transitions to the second midsection in a second direction.

80. The hemostatic valve of example 79, wherein the first midsection defines a width that is greater than the second midsection.

81. The hemostatic valve of any of examples 73–80, wherein the first sealing feature is a proximal sealing feature positioned at a first end of the unibody elongate member, and the second sealing feature is a distal sealing feature positioned at a second end of the unibody elongate member opposite from the first end.

82. The hemostatic valve of example 81, wherein the unibody elongate member comprises a midsection situated between the proximal sealing feature and the distal sealing feature.

83. The hemostatic valve of example 82, wherein the unibody elongate member is a multi-durometer elongate member, wherein the proximal and distal sealing features have a first durometer and the midsection has a second durometer.

84. The hemostatic valve of example 83, wherein the first durometer is in a range of about 40A to about 70A, and the second durometer is in the range of about 40A to about 70A.

85. The hemostatic valve of example 84, wherein the first durometer is about 70A and the second durometer is about 40A.

86. The hemostatic valve of example 83, wherein the first durometer is greater than the second durometer.

87. The hemostatic valve of example 83, wherein the first durometer and the second durometer are different.

88. The hemostatic valve of any of examples 73–87, wherein the unibody elongate member defines a thickness between about 0.005 inches and 0.05 inches.

89. A unibody elongate member configured for use within a hemostatic valve, comprising:

a proximal sealing feature positioned at a first end of the unibody elongate member;

a distal sealing feature positioned at a second end of the unibody elongate member opposite from the first end; and

a midsection positioned between the proximal sealing feature and the distal sealing feature,

wherein the proximal sealing feature defines a first cross-sectional width, the distal sealing feature defines a second cross-sectional width, and the midsection defines a third cross-sectional width that is smaller than the first and second cross-sectional widths, and

wherein the proximal sealing feature, the distal sealing feature, and the midsection define a continuous central lumen.

90. The unibody elongate member of example 89, wherein the unibody elongate member is a multi-durometer elongate member, wherein the proximal and distal sealing features have a first durometer, and the midsection has a second durometer.

91. The unibody elongate member of example 90, wherein the first durometer is in a range of about 40A to about 70A, and the second durometer is in the range of about 40A to about 70A.

92. The unibody elongate member of example 91, wherein the first durometer is about 70A and the second durometer is about 40A.

93. The unibody elongate member of example 90, wherein the first durometer is greater than the second durometer.

94. The unibody elongate member of example 90, wherein the first durometer and the second durometer are different.

95. The unibody elongate member of any of examples 89–94, wherein the midsection defines a straight tapered cross-section.

96. The unibody elongate member of example 95, wherein the midsection tapers a first distance from the first end in a first direction at a first angle, and the midsection tapers a second distance from the second end in a second direction at a second angle.

97. The unibody elongate member of any of examples 89–96, wherein the midsection defines a step-tapered cross-section.

98. The unibody elongate member of example 97, wherein the midsection defines a first midsection in proximity to the first end, wherein the first midsection transitions to a second midsection in a first direction, and the midsection defines the first midsection in proximity to the second end, wherein the first midsection transitions to the second midsection in a second direction.

99. The unibody elongate member of example 98, wherein the first midsection defines a width that is greater than the second midsection.

100. The unibody elongate member of any of examples 89–99, wherein the unibody elongate member defines a thickness between about 0.005 inches and 0.05 inches.

### V. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A hemostasis valve for sealing a medical device, the hemostasis valve comprising:
a unibody elongate member having a proximal sealing feature, a distal sealing feature, and a central lumen extending therebetween, wherein the unibody elongate member is pliable;
a tensioning mechanism including an actuator coupled to the unibody elongate member, wherein the actuator is moveable between (a) a first position wherein the central lumen is constricted and sealed, and (b) a second position wherein the central lumen is open, and wherein the tensioning mechanism comprises at least one filament extending at least partially around the unibody elongate member; and
a biasing member configured to bias the actuator to the first position.

2. The hemostasis valve of claim 1 wherein the proximal sealing feature and the distal sealing feature each include a flange.

3. The hemostasis valve of claim 2 wherein the flanges of the proximal sealing feature and the distal sealing feature are configured to mate with a proximal sealing feature and a distal sealing feature of a housing of the hemostasis valve, respectively.

4. The hemostasis valve of claim 1 wherein the unibody elongate member further comprises a midsection positioned between the proximal sealing feature and the distal sealing feature that defines a portion of the central lumen.

5. The hemostasis valve of claim 4 wherein the midsection includes a tubular wall defining the portion of the central lumen.

6. The hemostasis valve of claim 5 wherein a diameter of the tubular wall decreases toward a center of the midsection and wherein a rate of change of the diameter is constant along a length of the midsection.

7. The hemostasis valve of claim 5 wherein the midsection includes a first portion and a second portion, and wherein a first diameter of the tubular wall at the first portion is different than a second diameter of the tubular wall at the second portion.

8. The hemostasis valve of claim 7 wherein the tubular wall is either the first diameter or the second diameter along a majority of a length of the tubular wall.

9. The hemostasis valve of claim 5 wherein a distance between an inner surface and an outer surface of the tubular wall defines a thickness of the tubular wall, and wherein the thickness of the tubular wall varies along a length of the midsection.

10. An elongate member for use in a hemostasis valve, comprising:
a proximal sealing feature positioned at a first end of the elongate member;
a distal sealing feature positioned at a second end of the elongate member opposite from the first end; and
a midsection positioned between the proximal sealing feature and the distal sealing feature,
wherein the proximal sealing feature defines a first cross-sectional width, the distal sealing feature defines a second cross-sectional width, and the midsection defines a third cross-sectional width that is less than the first and second cross-sectional widths, and
wherein the proximal sealing feature, the distal sealing feature, and the midsection define a continuous central lumen.

11. The elongate member of claim 10 wherein a length of the midsection is greater than a length of the proximal sealing feature and a length of the distal sealing feature.

12. The elongate member of claim 10 wherein:
the midsection comprises a tubular member,
the proximal sealing feature comprises a proximal disc,
the distal sealing feature comprises a distal disc, and
the tubular member, the proximal disc, and the distal disc form a continuous integral structure.

13. The elongate member of claim 12 wherein the tubular member, the proximal disc, and the distal disc are configured to be complementary to features of a housing of a hemostasis valve.

14. The elongate member of claim 10 wherein a thickness of the proximal sealing feature, the distal sealing feature, and the midsection are equal.

15. A unibody elongate member configured for use within a hemostatic valve, comprising:
a proximal sealing feature;
a distal sealing feature; and
a midsection positioned between the proximal sealing feature and the distal sealing feature, wherein an outer diameter of the midsection is greater at an end of the midsection than at a center of the midsection, and the outer diameter of the midsection varies at a constant rate along a length of the midsection between the end and the center, and
wherein the proximal sealing feature, the distal sealing feature, and the midsection define a continuous central lumen.

16. The unibody elongate member of claim 15 wherein the proximal sealing feature and the distal sealing feature have a uniform thickness.

17. The unibody elongate member of claim 15 wherein a distance between an outer surface and an inner surface of the midsection varies between a first portion and a second portion of the midsection.

18. The unibody elongate member of claim 15 wherein:
the end of the midsection is a first end, the length of the midsection is first length, and the constant rate is a first constant rate;
the midsection further comprises a second end with an outer diameter greater than the outer diameter at the center of the midsection;
the outer diameter of the midsection varies at a second constant rate along a second length of the midsection between the second end and the center; and
the second constant rate is different than the first constant rate.

19. The unibody elongate member of claim 15 wherein a first durometer of the proximal sealing feature and the distal sealing feature is different than a second durometer of the midsection.

20. The unibody elongate member of claim 15 wherein the proximal sealing feature and the distal sealing feature each include a flange configured to mate with an inner channel of the hemostatic valve.
